(19) United States
(12) Patent Application Publication
(10) Pub. No.: US 2010/0130608 A1
(43) Pub. Date: May 27, 2010

(54) COMPOSITIONS AND METHODS FOR REDUCING TRIGLYCERIDE LEVELS

(75) Inventors: Alan Stuart RYAN, Ellicott City, MD (US); Edward B. Nelson, Austin, TX (US); Jung Lee, McLean, VA (US); Krishna Raman, Wilmington, DE (US)

Correspondence Address: Dechert LLP, 2440 W. El Camino Real, Suite 700, Mountain View, CA, 94040-1499, US

(73) Assignee: Martek Biosciences Corporation, Columbia, MD (US)

(21) Appl. No.: 12/572,263

(22) Filed: Oct. 1, 2009

Related U.S. Application Data
(60) Provisional application No. 61/101,973, filed on Oct. 1, 2008.

(51) Int. Cl. A61K 31/22; A61K 31/202; A61P 3/00

(52) U.S. Cl. 514/549; 514/560

(57) ABSTRACT

The present invention is directed to methods of reducing plasma triglyceride level in subjects by administering docosahexaenoic acid (DHA). The method can comprise administering daily to the subject a dosage form comprising docosahexaenoic acid ester substantially free of eicosapentaenoic acid (EPA), wherein the DHA is derived from an algal source. In some embodiments, the method comprises administering daily to the subject a dosage form comprising DHA ester substantially free of EPA, wherein the DHA ester is about 60% to about 99.5% (w/w) of the total fatty acid content of the dosage form. In some embodiments, the method comprises administering daily to the subject a dosage form comprising about 200 mg to about 4 g of DHA ester substantially free of EPA. In some embodiments, the foregoing methods also result in a lowering of the amount of total cholesterol in the subject.

### CROSS-REFERENCED APPLICATION

The present application claims benefit under 35 U.S.C. §119(e) of U.S. Provisional Patent Application Ser. No. 61/101,973, filed Oct. 1, 2008, the contents of which are incorporated herein by reference in their entirety.

### BACKGROUND OF THE INVENTION

1. Field of the Invention

The present invention is directed to methods of reducing plasma triglyceride level in subjects by administering docosahexaenoic acid (DHA). The methods can comprise administering daily to a subject a dosage form comprising docosahexaenoic acid ester substantially free of eicosapentaenoic acid (EPA), wherein the DHA is derived from an algal source. In some embodiments, the method comprises administering daily to a subject a dosage form comprising DHA ester substantially free of EPA, wherein the DHA ester is about 60% to about 99.5% (w/w) of the total fatty acid content of the dosage form. In some embodiments, the method comprises administering daily to a subject a dosage form comprising about 200 mg to about 4 g of DHA ester substantially free of EPA. In some embodiments, the foregoing methods also result in a lowering of the amount of total cholesterol in the subject.

2. Background Art

Triglycerides include a glycerol esterified to three fatty acids. In the human body, high levels of triglycerides in the bloodstream have been linked to atherosclerosis, and, by extension, the risk of heart disease and stroke, as well as diabetes mellitus, pancreatitis, chronic renal disease, and certain primary hyperlipidemias. High triglyceride levels have also been associated with obesity, depression, bipolar disorder, and other affective disorders. Glueck, C. J., et al., Am. J. Med. Sci. 308:218-225 (1994).

Clinical trials have demonstrated that omega-3 long-chain polyunsaturated fatty acids (LC-PUFA) lower triglyceride levels. Two particular polyunsaturated fatty acids that have been shown to have therapeutic efficacy of reducing triglyceride levels when used in combination include (all-Z)5,8,11,14,17-eicosapentaenoic acid, hereinafter referred to as EPA, and (all-Z)-4,7,10,13,16,19-docosahexaenoic acid, hereinafter referred to as DHA. EPA is known to be a precursor in the biosynthesis of prostaglandin PGE₃. These LC-PUFA are commonly found together in fatty fish, such as tuna, salmon, and mackerel.

Early studies of LC-PUFAs focused primarily on the effects of EPA on triglyceride levels. The relative contribution of other LC-PUFAs remained to be defined. Additional data, however, demonstrate that DHA and EPA have important cardioprotective properties. See, e.g., Mori and Holub, J. Nutr 126:3032-3039 (1996).

Previously, it was difficult to obtain pure EPA and DHA since the main source of these fatty acids, which occur together, was from the fats and oils of fish and marine animals. Unfortunately, in these sources, other fatty acids were always present in larger amounts. Most methods for extracting EPA and DHA from other triglycerides have not been satisfactory for producing high purity fatty acids, thereby making clinical studies difficult to conduct.

### BRIEF SUMMARY OF THE INVENTION

The present invention is directed to a method of reducing plasma triglyceride levels in a subject by administering DHA. The method can comprise administering daily to a subject a dosage form comprising docosahexaenoic acid (DHA) ester substantially free of eicosapentaenoic acid (EPA), wherein the DHA ester is derived from an algal source. In some embodiments, the method comprises administering daily to the subject a dosage form comprising docosahexaenoic acid ester substantially free of eicosapentaenoic acid, wherein the DHA ester is about 60% to about 99.5% (w/w) of the total fatty acid content of the dosage form, and wherein the DHA ester is derived from an algal source. In some embodiments, the method comprises administering daily to the subject a dosage form comprising about 200 mg to about 4 g of DHA ester substantially free of EPA. In some embodiments, the foregoing methods also result in a lowering of the amount of total cholesterol in the subject.

In some embodiments of the invention, the DHA ester is a DHA alkyl ester, e.g., a DHA methyl ester, ethyl ester or propyl ester. The DHA ester can be derived from various sources. In some embodiments, the DHA ester is derived from an algal source, e.g., Crypthecodinium cohnii or Schizochytrium sp.

The DHA ester used in the methods of the present invention can be purified to various levels. In some embodiments, the DHA ester is about 60% to about 99.5% (w/w) of the total fatty acid content of the dosage form, or about 85% to about 95% (w/w) of the total fatty acid content of the dosage form. In some embodiments, the dosage form comprises about 0.5 g to about 4 g of DHA ester, or about 1 g to about 2 g of DHA ester.

The methods of the present invention use a dosage form substantially free of EPA. In some embodiments, the EPA is less than 1% of the total fatty acid content of the dosage form, less than 0.2% of the total fatty acid content of the dosage form, or less than 0.01% of the total fatty acid content of the dosage form.

In some embodiments, additional fatty acids are present in the dosage form. For example, in some embodiments, the dosage form comprises 0.1% to 20% or about 0.1% to about 20% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) a-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises 1% to 5% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises less than 1% each of the following fatty acids: (a) capric acid; (b) lauric acid; (c) myristic acid; (d) palmitic acid; (e) palmitoleic acid; (f) stearic acid; (g) oleic acid; (h) linoleic acid; (i) a-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form comprises 4,7,10,13,16,19,22,25 octacosaoctaenoic acid (C28:8) in an amount of from about 0.5% to about 3%, from about 1% to about 2%, or about 1.3% (w/w) of the total fatty acid content of the dosage form.

In some embodiments, the invention is directed to a method of reducing plasma triglyceride levels in a subject, wherein the subject has a chronic condition, e.g., chronic elevated triglyceride levels. Thus, the invention can be directed to administering daily to the subject a dosage form comprising DHA ester for the remainder of the subject's lifetime (i.e., chronic administration), from 1 year to 20 years, or from 1 year to 10 years. In some embodiments, the invention is directed to a method of reducing plasma triglyceride level in a subject, the method comprising administering daily to the subject a dosage form comprising about 200 mg to about 4 g of DHA ester substantially free of EPA. The dosage form can be administered daily for 4 to 28 consecutive days, or for 7 to 14 consecutive days. In certain aspects of the foregoing embodiments, the administration of DHA ester results in a reduction in the subject's total cholesterol levels. The triglyceride levels and/or cholesterol levels in a subject can be reduced relative to a subject that has not been administered a dosage form comprising DHA ester. For example, in some embodiments the triglyceride levels in a subject are reduced about 25% to about 75% by day 14, or about 30% to about 65% by day 14, relative to a subject that has not been administered a dosage form comprising DHA ester. In certain aspects of those embodiments in which the subject's total cholesterol is also lowered, the total cholesterol level in a subject is reduced by about 15%, about 20%, about 25%, about 40%, about 15% to about 25% by day 14, or about 20% to about 40% by day 28, relative to a subject that has not been administered a dosage form comprising DHA ester.

The methods of the present invention can include administration of the dosage form once daily. In some embodiments, the dosage form is an oral dosage form, e.g., a tablet, pill, gel cap or caplet.

The present invention is also directed to an oral dosage form comprising: (a) about 200 mg to about 3 g of DHA ester; wherein the DHA ester is about 60% to about 99.5% (w/w) of the total fatty acid content of the dosage form; (b) a pharmaceutically acceptable excipient; wherein the dosage form is substantially free of EPA, and wherein the DHA ester is derived from an algal source.

### BRIEF SUMMARY OF THE FIGURES

FIG. 1 represents the plasma DHA fatty acid area percent in rats fed either DHA-EE, DHASCO®, or Lovaza®.

FIG. 2 represents the mean DHA levels from plasma total lipids assays. The DHA-EE administered was prepared as in Example 1.

FIG. 3 represents the plasma EPA fatty acid area percent in rats fed either DHA-EE, DHASCO®, or Lovaza®.

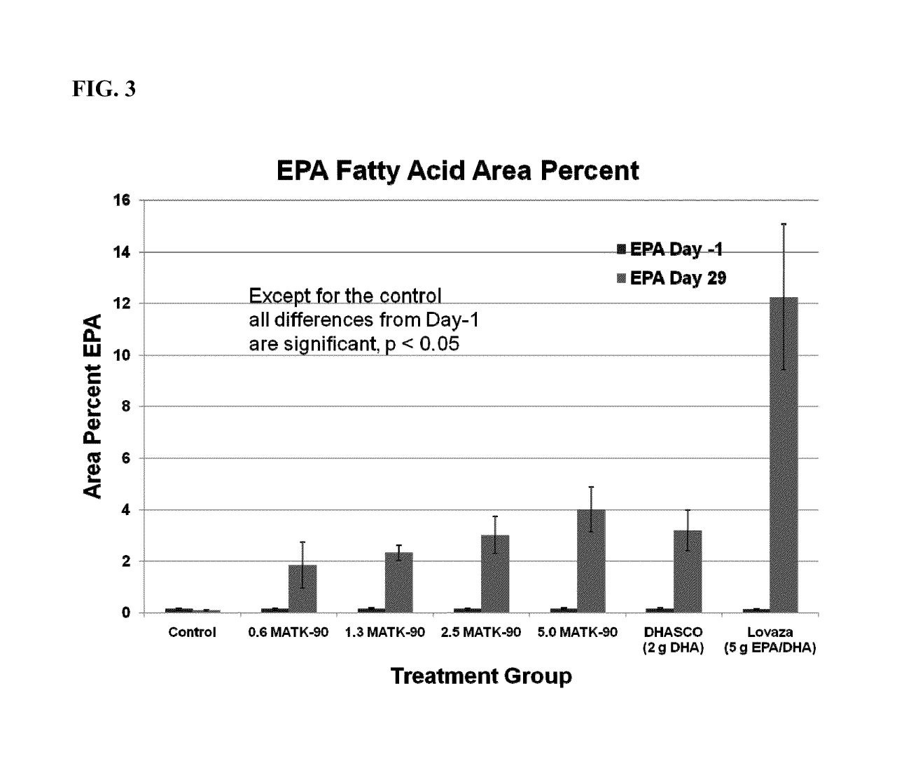

FIG. 4 represents the mean EPA levels from plasma total lipids assays. The DHA-EE administered was prepared as in Example 1.

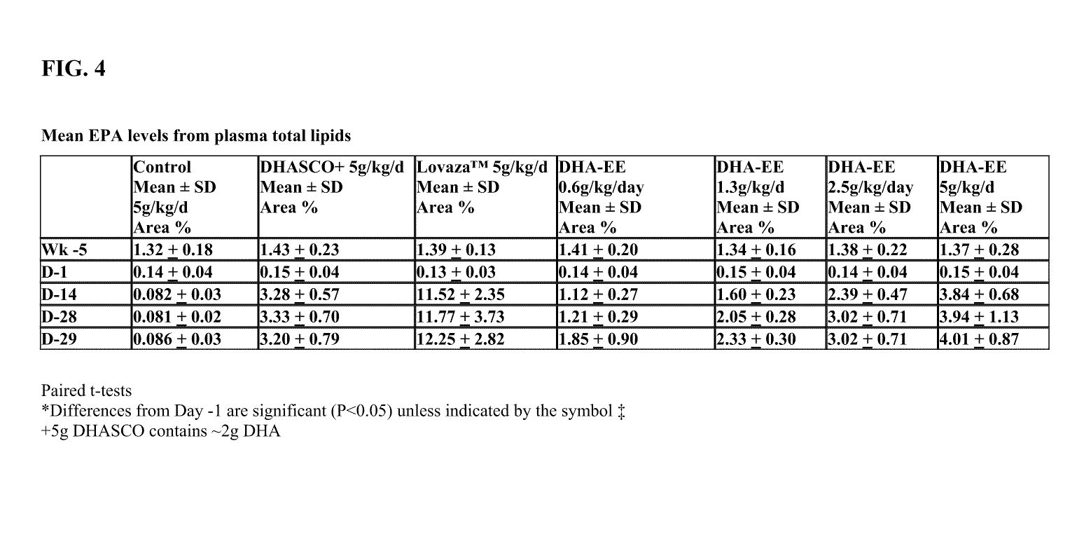

FIG. 5 represents a regression analysis of absolute change from baseline in triglyceride levels versus baseline triglyceride levels, wherein 1 to 6 g/day of DHA ethyl ester as prepared in Example 1 was used.

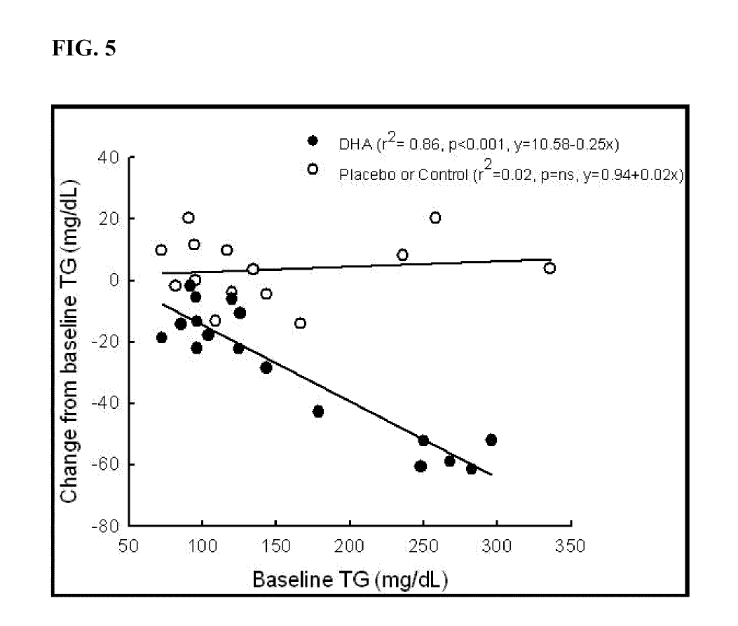

### DETAILED DESCRIPTION OF THE INVENTION

The present invention is directed to methods of reducing plasma triglyceride level in a subject using DHA ester. The term DHA refers to docosahexaenoic acid, also known by its chemical name (all-Z)-4,7,10,13,16,19-docosahexaenoic acid. DHA is an ω-3 polyunsaturated fatty acid. In some embodiments, the foregoing methods also result in a lowering of the amount of total cholesterol levels in the subject.

The DHA of the present invention is an ester. The term “ester” refers to the replacement of the hydrogen in the carboxylic acid group of the DHA molecule with another substituent. Typical esters are known to those in the art, a discussion of which is provided by Higuchi, T. and V. Stella in Pro-drugs as Novel Delivery Systems, Vol. 14, A.C.S. Symposium Series, Bioreversible Carriers in Drug Design, Ed. Edward B. Roche, American Pharmaceutical Association, Pergamon Press, 1987, and Protective Groups in Organic Chemistry, McOmie ed., Plenum Press, New York, 1973. Examples of the most common esters include methyl, ethyl, propyl, butyl, pentyl, t-butyl, benzyl, nitrobenzyl, methoxybenzyl, benzhydryl, and trichloroethyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-(ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a linear or cyclic hydrocarbon group, e.g., a C₁-C₆ alkyl, C₁-C₆ cycloalkyl, C₁-C₆ alkenyl, or C₁-C₆ aryl ester. In some embodiments, the added substituent is a Cᵢ, C₂, C₃, C₄, C₅ or a C₆ alkyl. In some embodiments, the ester is an alkyl ester, e.g., a methyl ester, ethyl ester or propyl ester. In some embodiments, the ester is an ethyl ester or a DHA-ethyl ester. In some embodiments, the ester substituent is added to the DHA free acid molecule when the DHA is in a purified or semi-purified state. Alternatively, the DHA ester is formed upon conversion of a triglyceride to an ester. One of skill in the art can appreciate that some non-esterified DHA molecules may be present in the present invention, e.g., DHA molecules that have not been esterified, or DHA ester linkages that have been cleaved, e.g., hydrolyzed. In some embodiments, the non-esterified DHA molecules constitute less than 3% (mol/mol), about 2% to about 0.01% (mol/mol), about 1% to about 0.05% (mol/mol), or about 5% to about 0.1% (mol/mol) of the total DHA molecules. Alternatively, in some embodiments, the DHA of the present invention can be in a free acid form and/or in a salt form.

The DHA ester of the present invention can be derived from various sources, e.g., from oleaginous microorganisms. As used herein, “oleaginous microorganisms” are defined as microorganisms capable of accumulating greater than 20% of the dry weight of their cells in the form of lipids. In some embodiments, the DHA ester is derived from a phototrophic or heterotrophic single cell organism or multicellular organism, e.g., an algae. For example, the DHA can be derived from a diatom, e.g., a marine dinoflagellates (algae), such as Crypthecodinium sp., Thraustochytrium sp., Schizochytrium sp., or combinations thereof.

The source of the DHA can include a microbial source, including the microbial groups Stramenopiles, Thraustochytrids, and Labrinthulids. Stramenopiles includes microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), and Chlamydomyxa* (species include labyrinthuloides, montana) (*=there is no current general consensus on the exact taxonomic placement of these genera).

In some embodiments, the algal source is, e.g., Crypthecodinium cohnii. Samples of C. cohnii, have been deposited with the American Type Culture Collection at Rockville, Md., and assigned the accession numbers 40750, 30021, 30334-30348, 30541-30543, 30555-30557, 30571, 30572, 30772-30775, 30812, 40750, 50050-50060, and 50297-50300.

As used herein, the term microorganism, or any specific type of organism, includes wild strains, mutants or recombinant types. Organisms which can produce an enhanced level of oil containing DHA are considered to be within the scope of this invention. Also included are microorganisms designed to efficiently use more cost-effective substrates while producing the same amount of DHA as the comparable wild-type strains. Cultivation of dinoflagellates such as C. cohnii has been described previously. See, U.S. Pat. No. 5,492,938 and Henderson, et al., Phytochemistry 27:1679-1683 (1988). Organisms useful in the production of DHA can also include any manner of transgenic or other genetically modified organisms, e.g., plants, grown either in culture fermentation or in crop plants, e.g., cereals such as maize, barley, wheat, rice, sorghum, pearl millet, corn, rye and oats; or beans, soybeans, peppers, lettuce, peas, Brassica species (e.g., cabbage, broccoli, cauliflower, brussel sprouts, rapeseed, and radish), carrot, beets, eggplant, spinach, cucumber, squash, melons, cantaloupe, sunflowers, safflower, canola, flax, peanut, mustard, rapeseed, chickpea, lentil, white clover, olive, palm, borage, evening primrose, linseed, and tobacco.

DHA ester can be purified to various levels. DHA purification can be achieved by any means known to those of skill in the art, and can include a method comprising: a) reacting the composition in the presence of an alcohol and a base to produce an ester of a polyunsaturated fatty acid from the triglycerides; and b) distilling the composition to recover a fraction comprising the ester of the polyunsaturated fatty acid, optionally wherein the method further comprises: c) combining the fraction comprising the ester of the polyunsaturated fatty acid with urea in a medium; d) cooling or concentrating the medium to form a urea-containing precipitate and a liquid fraction; and e) separating the precipitate from the liquid fraction. See, e.g., U.S. application Ser. No. 12/163,555, filed Jun. 27, 2008, by Raman et al. titled “Production and Purification of Esters of Polyunsaturated Fatty Acids,” incorporated by reference herein in its entirety and published on Jan. 22, 2009 as 2009/0023808 A1. In some embodiments, the purification process includes starting with refined, bleached, and deodorized oil (RBD oil), then performing low temperature fractionation using acetone to provide a concentrate. The concentrate can be obtained by base-catalyzed transesterification, distillation, and silica refining to produce the final DHA product.

Means of determining purity levels of fatty acids are known in the art, and can include, e.g., chromatographic methods such as, e.g., HPLC silver ion chromatographic columns (ChromSpher 5 Lipids HPLC Column, Chrompack, Raritan N.J.). Alternatively, the purity level can be determined by gas chromatography, with or without converting DHA to the corresponding methyl ester. In some embodiments, the DHA ester is greater than 60% (w/w) of the total fatty acid content of the dosage form, about 70% to 99.9% (w/w) of the total fatty acid content of the dosage form, about 80% to about 99.5% (w/w) of the total fatty acid content of the dosage form, about 82% to about 99% (w/w) of the total fatty acid content of the dosage form, or about 85% to about 98% (w/w) of the total fatty acid content of the dosage form. In some embodiments, the DHA ester is greater than about 85%, about 87%, about 90%, about 92% or about 95% (w/w) of the total fatty acid content of the dosage form. In some embodiments, the percentage of DHA ester in a dosage form is standardized to a desired total fatty acid content, such as the foregoing DHA ester percentages and ranges. For example, in some embodiments, a non-polyunsaturated fatty acid, such as oleic acid, is used to dilute the DHA ester content to a standardized DHA ester content of about 90% (w/w) of the total fatty acid content of the dosage form.

The methods of the present invention use dosage forms substantially free of EPA. The term EPA refers to the free eicosapentaenoic acid, know by its chemical name (all-Z)5,8,11,14,17-eicosapentaenoic acid, as well as any salts or esters thereof. Thus, the term “EPA” would encompass the free acid EPA as well as EPA ethyl esters and triglycerides containing EPA. EPA can be removed during the purification of the DHA ester, or alternatively, the DHA ester can be derived from an organism that does not produce EPA, or produces very little EPA. In some embodiments, the term “substantially free of EPA” means EPA is less than 3% of the total fatty acid content of the dosage form, less than 2% of the total fatty acid content of the dosage form, less than 1% of the total fatty acid content of the dosage form, less than 0.5% of the total fatty acid content of the dosage form, less than 0.2% of the total fatty acid content of the dosage form, or less than 0.01% of the total fatty acid content of the dosage form. In some embodiments, the EPA is not detected in the dosage forms using techniques currently known in the art.

In the present invention, additional fatty acids can be present in the dosage forms. These fatty acids can include fatty acids that were not removed during the purification process, i.e., fatty acids that were co-isolated with DHA from an organism. These fatty acids can be present in various concentrations. For example, in some embodiments, the dosage form comprises 0.1% to 20% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) a-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, a dosage form comprises 1.0% to 5% of one or more of the following fatty acids: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, a dosage form comprises less than 1% each of the following fatty acids: (a) capric acid; (b) lauric acid; (c) myristic acid; (d) palmitic acid; (e) palmitoleic acid; (f) stearic acid; (g) oleic acid; (h) linoleic acid; (i) a-linolenic acid; (j) docosapentaenoic acid 22:5n-3, 22:5w3 (DPAn3); (k) docosapentaenoic acid 22:5n-6, 22:5w6 (DPAn6); and (l) 4,7,10,13,16,19,22,25 octacosaoctaenoic acid (C28:8). In some embodiments, the dosage form of the present invention does not contain a measurable amount of docosapentaenoic acid 22:5n-3, 22:5w3 (DPAn3); docosapentaenoic acid 22:5n-6, 22:5w6 (DPAn6); and/or 4,7,10,13,16,19,22,25 octacosaoctaenoic acid (C28:8). In some embodiments, the dosage form comprises 4,7,10,13,16,19,22,25 octacosaoctaenoic acid (C28:8) in an amount of from about 0.5% to about 3%, from about 1% to about 2%, or about 1.3% (w/w) of the total fatty acid content of the dosage form. In some embodiments, the dosage form comprises 4,7,10,13,16,19,22,25 octacosaoctaenoic acid (C28:8) in an amount of about 1.3% (w/w) of the total fatty acid content of the dosage form.

Various dosage amounts of DHA ester can be administered to a subject. The terms “daily dosage,” “daily dosage level,” and “daily dosage amount” refer to the total amount of DHA ester administered per day (about 24 hour period). Thus, for example, administration of DHA ester to a subject at a daily dosage of 2 g means that the subject receives a total of 2 g of DHA ester on a daily basis, whether the DHA ester is administered as a single dosage form comprising 2 g DHA ester, or alternatively, four dosage forms comprising 500 mg DHA ester each (for a total of 2 g DHA ester). In some embodiments, the daily amount of DHA ester is administered in a single dosage form, or in two or more dosage forms. The dosage forms of the present invention can be taken in a single application or multiple applications per day. For example, if four capsules are taken daily, each capsule comprising 500 mg DHA ester, then all four capsules could be taken once daily, or 2 capsules could be taken twice daily, or 1 capsule could be taken every 6 hours. In some embodiments, the daily amount of DHA ester is less than about 4 g, about 200 mg to about 3.8 g, about 500 mg to about 3.7 g, about 750 mg to about 3.5 g, or about 1 g to about 2 g DHA ester. In some embodiments, the daily amount of DHA ester is about 520 mg to about 4 g, about 540 mg to about 4 g, about 560 mg to about 4 g, or about 580 mg to 4 g. In some embodiments, the daily amount of DHA ester is less than about 3.8 g DHA ester, about 900 mg to about 3.6 g DHA ester, or about 1.8 g to about 2.7 g of DHA ester. In some embodiments, the daily amount of DHA ester comprises about 450 mg, 500 mg, 520 mg, 540 mg, 600 mg, 700 mg, 800 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA ester. In some embodiments, the daily amount of DHA ester is within a range, inclusive or exclusive of endpoints, in which the upper and lower limits are independently selected from the following amounts: 450 mg, 500 mg, 520 mg, 540 mg, 600 mg, 700 mg, 800 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, and 10 g.

Various dosage amounts of DHA ester can be in a dosage form. In some embodiments, the dosage form comprises less than about 4 g of DHA ester, less than about 3 g of DHA ester, about 100 mg to about 3.8 g DHA ester, about 200 mg to about 3.6 g of DHA ester, about 500 mg to about 3.5 g DHA ester, or about 1 g to about 2.0 g DHA ester. In some embodiments, the dosage form comprises less than about 4 g of DHA ester, less than about 3 g of DHA ester, about 200 mg to about 3.9 g DHA ester, about 500 mg to about 3.7 g of DHA ester, about 750 mg to about 3.5 g DHA ester, about 750 mg to about 3.0 g DHA ester, or about 1 g to about 2 g DHA ester. In some embodiments, the dosage form comprises less than about 3.8 g DHA ester, about 900 mg to about 3.6 g DHA ester, or about 1.8 g to about 2.7 g of DHA ester. In some embodiments, the dosage form comprises about 200 mg, 450 mg, 500 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA ester. In some embodiments, the amount of DHA ester in the dose or dosage form is within a range, inclusive or exclusive of endpoints, in which the upper and lower limits are independently selected from the following amounts: 200 mg, 450 mg, 500 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA ester. In other embodiments, the amount of DHA ester in the dose or dosage form is about 200 mg, 450 mg, 500 mg, 900 mg, 1 g, 1.5 g, 1.8 g, 2.0 g, 2.5 g, 2.7 g, 3.0 g, 3.2 g, 3.3 g, 3.4 g, 3.5 g, 3.6 g, 3.7 g, 3.8 g, 3.9 g, 4.0 g, 4.5 g, 5.0 g, 6.0 g, 6.5 g, 7 g, 8 g, 9 g, or 10 g DHA ester.

The present invention is directed to methods of lowering plasma triglyceride levels. In some embodiments, the methods also result in a lowering of the amount of total cholesterol in the subject. The measurement of plasma triglyceride levels and total cholesterol can be accomplished using any of the commercially available devices, e.g., a Vitros 750 (Johnson & Johnson, Ortho Clinical Diagnostics, Raritan, N.J.), or an Olympus AU640™ Chemistry Immune Analyzer (Center Valley, Pa.). The term “lowering” refers to the reduction of plasma triglyceride levels in a subject, wherein the triglyceride levels of the subject are measured before and after administration of the DHA ester dosage form. As one of skill in the art will recognize, triglyceride levels fluctuate in a subject depending on many factors, e.g., diet (e.g., fasting conditions), exercise regimens, time of day, etc. Thus, the term “lowering” refers to the relative amounts of triglyceride levels of a subject before and after administration of DHA ester, wherein the diet, exercise regimens and time of day are controlled. Similarly, the term “lowering” total cholesterol refers to the reduction of total cholesterol in a subject, wherein the total cholesterol levels of the subject are measured before and after administration of the DHA ester dosage form. As one of skill in the art will recognize, total cholesterol levels fluctuate in a subject depending on many factors, e.g., diet (e.g., fasting conditions), exercise regimens, time of day, etc. Thus, the term “lowering” refers to the relative total cholesterol levels of a subject before and after administration of DHA ester, wherein the diet, exercise regimens and time of day are controlled.

The triglyceride levels in a subject can be reduced relative to a subject that has not been administered a dosage form comprising DHA ester. In some embodiments of the present invention, the triglyceride levels are reduced greater than 5%, or about 5% to about 90%, about 10% to about 80%, about 25% to about 75%, or about 30% to about 65%. In some instances, the triglyceride levels in a subject can be reduced relative to a subject that has been administered a dosage form comprising DHA and EPA, e.g., Lovaza®. In these instances, the triglyceride levels can be reduced greater than 5%, or about 5% to about 90%, about 10% to about 80%, about 25% to about 75%, or about 30% to about 65% relative to a subject administered DHA and EPA. One of skill in the art will appreciate that the amount of the reduction can be dependent on the initial triglyceride level in the subject. For example, in subjects having a higher original triglyceride level, the amount of triglyceride reduction can be greater, relative to a subject with a lower original triglyceride level. Triglyceride levels reduction can also be dependent on the length and/or amount of administration of DHA ester, or the regimen of administration of the DHA ester. For example, in some embodiments, the subject has a chronic condition, and is administered the DHA ester of the present invention for the remainder of the subject's lifetime, or from 1 to 20 years, or 1, 2, 5, 10, or 15 years. In some embodiments, the triglyceride levels in a subject are reduced by greater than 5%, about 5% to about 90%, about 25% to about 75%, or about 30% to about 65% by year 1, 5, 10, 15 or 20 years. In certain embodiments, the triglyceride level in a subject is reduced by a percentage that is within a range, inclusive or exclusive of endpoints, wherein the upper and lower limits of the range are independently selected from the following amounts: about 5%, about 10%, about 15%, about 20%, about 25%, about 30%, about 35%, about 40%, and about 50%; about 60%, about 70%, about 80% and about 90%; optionally within a time period selected from the following times periods: about 7 days, about 2 weeks, about 1 month, about 6 weeks, about 2 months, about 3 months, about 6 months, about 1 year, about 5 years.

In those embodiments that also result in a lowering of the amount of total cholesterol in the subject, the amount of total cholesterol in the subject will be reduced relative to a subject that has not been administered a dosage form comprising DHA ester. In some embodiments of the present invention, the total cholesterol levels are reduced by about 15%, about 20%, about 25%, about 40%, about 15% to about 25%, or about 20% to about 40%. In certain embodiments in which the subject's total cholesterol is lowered, the total cholesterol level in a subject is reduced by a percentage that is within a range, inclusive or exclusive of endpoints, wherein the upper and lower limits of the range are independently selected from the following amounts: about 10%, about 15%, about 20%, about 25%, about 30%, about 35%, about 40%, and about 50%; optionally within a time period selected from the following times periods: about 7 days, about 2 weeks, about 1 month, about 6 weeks, about 2 months, about 3 months, about 6 months, about 1 year, about 5 years. In some instances, the total cholesterol levels in a subject can be reduced relative to a subject that has been administered a dosage form comprising DHA and EPA, e.g., Lovaza®. One of skill in the art will appreciate that the amount of the reduction can be dependent on the initial total cholesterol level in the subject. For example, in subjects having a higher original total cholesterol levels, the amount of total cholesterol reduction can be greater, relative to a subject with a lower original total cholesterol level. Total cholesterol level reduction can also be dependent on the length and/or amount of administration of DHA ester, or the regimen of administration of the DHA ester. For example, in some embodiments, the subject has a chronic condition, and is administered the DHA ester of the present invention for the remainder of the subject's lifetime, or from 1 to 20 years, or 1, 2, 5, 10, or 15 years.

In some embodiments, DHA ester of the present invention is administered daily for a shorter duration, e.g., 1 week to 12 weeks (week 1 to week 12). In some embodiments, the triglyceride levels in a subject are reduced by greater than 5%, about 5% to about 90%, about 25% to about 75%, or about 30% to about 65% on week 12. In those embodiments that also result in a lowering of the amount of total cholesterol in the subject, the total cholesterol levels in the subject are also reduced by about 15%, about 20%, about 25%, about 40%, about 15% to about 25%, or about 20% to about 40% on week 12. In some embodiments, the DHA ester is administered daily for 1 week to 6 weeks (week 1 to week 6). In some embodiments, the triglyceride levels in a subject are reduced by greater than 5%, about 5% to about 90%, about 25% to about 75%, or about 30% to about 65% on week 6. In those embodiments that also result in a lowering of the amount of total cholesterol in the subject, the total cholesterol levels in a subject are also reduced by about 15%, about 20%, about 25%, about 40%, about 15% to about 25%, or about 20% to about 40% on week 6. In some embodiments, the DHA ester is administered daily for 2 weeks to 4 weeks (week 2 to week 6). In some embodiments, the triglyceride levels in a subject are reduced by greater than 5%, about 5% to about 90%, about 25% to about 75%, or about 30% to about 65% on week 6. In some embodiments, the total cholesterol levels in a subject are also reduced by about 15%, about 20%, about 25%, about 40%, about 15% to about 25%, or about 20% to about 40% on week 6. In some embodiments, the DHA ester is administered daily for 28 days (day 28). In some embodiments, the triglyceride levels in a subject are reduced by greater than 5%, about 5% to about 90%, about 25% to about 75%, or about 30% to about 65% by day 28. In some embodiments, the total cholesterol levels in a subject are also reduced by about 15%, about 20%, about 25%, about 40%, about 15% to about 25%, or about 20% to about 40% by day 28. In some embodiments, the DHA ester is administered daily for 14 days (day 14). In some embodiments, the triglyceride levels in a subject are reduced by greater than 5%, about 5% to about 90%, about 25% to about 75%, or about 30% to about 65% by day 14. In some embodiments, the total cholesterol levels in a subject are also reduced by about 15%, about 20%, about 25%, about 40%, about 15% to about 25%, or about 20% to about 40% by day 14. In some embodiments, the DHA ester is administered daily for 7 days (day 7). In some embodiments, the triglyceride levels in a subject are reduced greater than 5%, about 5% to about 90%, about 25% to about 75%, or about 30% to about 65% by day 7. In some embodiments, the total cholesterol levels in a subject are also reduced by about 15%, about 20%, about 25%, about 40%, about 15% to about 25%, or about 20% to about 40% by day 7.

In some embodiments of the present invention, the DHA ester provides a rapid onset of triglyceride level reduction, relative to administration of (a) a composition comprising DHA and EPA, (b) triglyceride form of DHA, and/or (c) an impure form of DHA (e.g., a composition wherein <79% of the total fatty acid composition is DHA). In some embodiments, the impure form of DHA comprises a composition wherein <60%, <70%, <80%, <85%, <90%, <95%, <96%, <97%, <98%, <99%, <99.5%, <99.8% or <99.9% of the total fatty acid composition is DHA. The term “rapid onset” refers to the reduced time needed to lower a subject's triglyceride level to a designated point. For example, in some embodiments, the DHA ester of the present invention reduces the triglyceride level 5%, 10%, 15%, 20%, 30%, 40%, or 50% faster than a composition comprising (a) DHA and EPA, (b) triglyceride form of DHA, and/or (c) an impure form of DHA (e.g., a composition wherein <79% of the total fatty acid composition is DHA). Similarly, in certain embodiments that also result in a lowering of the amount of total cholesterol in the subject, the DHA ester provides a rapid onset of total cholesterol reduction.

The method of the present invention can be administered to individuals who have normal triglyceride levels (under 150 mg triglyceride/dL), or elevated triglyceride levels, e.g., borderline high triglyceride levels (151-200 mg triglyceride/dL), high triglyceride levels (201-499 mg triglyceride/dL), or very high triglyceride levels (hypertriglyceridemia) (>500 mg triglyceride/dL). Thus, in some embodiments the invention is directed to a method of treating a subject having normal triglyceride levels, borderline high triglyceride levels, high triglyceride levels, or very high triglyceride levels, the method comprising administration of the DHA esters as described herein. In some embodiments, the DHA esters as described herein are administered as an adjunct to diet to reduce triglyceride levels. In certain embodiments, the subject is an adult subject with very high triglyceride levels, i.e., hypertriglyceridemia. In some embodiments, the present invention is directed to methods of treating hypertriglyceridemia (elevated triglyceride levels), comprising administration of DHA esters as described herein. Hypertriglyceridemia can include familial hypertriglyceridemia. In some embodiments, the method of the present invention can be used to treat chronic elevated (i.e., borderline high triglyceride levels, high triglyceride levels, or very high triglyceride levels) triglyceride levels for the remainder of the life of the subject.

In some embodiments, the subject has normal total cholesterol levels (under 200 mg/dL), or elevated total cholesterol, e.g., borderline total cholesterol levels (200-239 mg/dL) or high total cholesterol levels (240 mg/dL or greater). In some embodiments, the methods of the invention result in a lowering of a subject's total cholesterol from high total cholesterol to borderline or normal total cholesterol levels. In some embodiments, the methods of the invention result in a lowering of a subject's total cholesterol from borderline to normal total cholesterol.

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms associated with elevated triglyceride levels; diminishment of the extent of the condition associated with elevated triglyceride levels; stabilization (i.e., not worsening) of the state of the condition, disorder or disease associated with elevated triglyceride levels; delay in onset or slowing of the condition, disorder or disease progression associated with elevated triglyceride levels; amelioration of the condition, disorder or disease state, remission (whether partial or total) the condition, disorder or disease associated with elevated triglyceride levels, whether detectable or undetectable; or enhancement or improvement of the condition, disorder or disease associated with triglyceride levels. In those embodiments that also result in a lowering of the amount of total cholesterol in the subject, the beneficial or desired clinical results include, but are not limited to, alleviation of symptoms associated with elevated total cholesterol levels; diminishment of the extent of the condition associated with elevated total cholesterol levels; stabilization (i.e., not worsening) of the state of the condition, disorder or disease associated with elevated total cholesterol levels; delay in onset or slowing of the condition, disorder or disease progression associated with elevated total cholesterol levels; amelioration of the condition, disorder or disease state, remission (whether partial or total) the condition, disorder or disease associated with elevated total cholesterol levels, whether detectable or undetectable; or enhancement or improvement of the condition, disorder or disease associated with total cholesterol levels. Treatment includes eliciting a clinically significant response, without excessive levels of side effects. Treatment also includes prolonging survival as compared to expected survival if not receiving treatment.

Triglycerides and cholesterol can be associated in lipoprotein complexes in the bloodstream, and can be separated into various densities, e.g., high-density lipoproteins (HDL), intermediate-density lipoproteins (IDL), low-density lipoproteins (LDL), and very low-density lipoproteins (VLDL). High cholesterol, LDL and VLDL levels have been correlated with development of atherosclerosis, cardiovascular morbidity, and mortality in humans. Thus, in some embodiments, the present invention is directed to methods of reducing LDL or VLDL levels in a subject, the methods comprising administration of the DHA ester dosages of the present invention. The invention is directed to methods of reducing, preventing, or slowing the development of atherosclerosis, cardiovascular morbidity, and/or mortality in humans comprising administration of the DHA ester dosages of the present invention. The term “preventing” means to stop or hinder a disease, disorder, or symptom of a disease or condition.

The term “subject” refers to mammals such as humans or primates, such as apes, monkeys, orangutans, baboons, gibbons, and chimpanzees. The term “subject” can also refer to companion animals, e.g., dogs and cats; zoo animals; equines, e.g., horses; food animals, e.g., cows, pigs, and sheep; and disease model animals, e.g., rabbits, mice, and rats. The subject can be a human or non-human. The subject can be of any age. For example, in some embodiments, the subject is a human infant, i.e., post natal to about 1 year old; a human child, i.e., a human between about 1 year old and 12 years old; a pubertal human, i.e., a human between about 12 years old and 18 years old; or an adult human, i.e., a human older than about 18 years old. In some embodiments, the subject is an adult, either male or female.

In some embodiments, the subject is a “subject in need thereof” A subject in need thereof refers to an individual for whom it is desirable to treat, i.e., to lower triglyceride levels, prevent increased triglyceride levels, retard increased triglyceride levels, or reduce the increase of triglyceride levels. Subjects in need thereof can also include subjects presenting with the effects of elevated triglyceride levels, e.g., subjects suffering from atherosclerosis, heart disease, stroke, diabetes mellitus, pancreatitis, chronic renal disease, and various hyperlipidemias.

“Pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compounds (e.g., DHA ester), compositions, and dosage forms of the present invention are pharmaceutically acceptable.

In some embodiments, the DHA ester is administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three times daily, as long as the dosage levels as specified herein are not exceeded.

The DHA ester can be formulated in a dosage form. These dosage forms can include, but are not limited to, tablets, capsules, cachets, pellets, pills, gel caps, powders and granules; and parenteral dosage forms which include, but are not limited to, solutions, suspensions, emulsions, coated particles, and dry powder comprising an effective amount of the DHA ester as taught in this invention. Various substances are known in the art to coat particles, including cellulose derivatives, e.g., microcrystalline cellulose, methyl cellulose, carboxymethyl cellulose; polyalkylene glycol derivatives, e.g., polyethylene glycol; talc, starch, methacrylates, etc. In some embodiments, the dosage form is a capsule, wherein the capsule is filled with a solution, suspension, or emulsion comprising the DHA ester. It is also known in the art that the active ingredients can be contained in such formulations with pharmaceutically acceptable excipients such as diluents, fillers, disintegrants, binders, lubricants, surfactants, hydrophobic vehicles, water soluble vehicles, emulsifiers, buffers, humectants, moisturizers, solubilizers, preservatives, flavorants, taste-masking agents, sweeteners, and the like. Suitable excipients can include, e.g., vegetable oils (e.g., corn, soy, safflower, sunflower, or canola oil). In some embodiments, the preservative can be an antioxidant, e.g., sodium sulfite, potassium sulfite, metabisulfite, bisulfites, thiosulfates, thioglycerol, thiosorbitol, cysteine hydrochloride, α-tocopherol, and combinations thereof. The means and methods for administration are known in the art and an artisan can refer to various pharmacologic references for guidance. For example, “Modern Pharmaceutics,” Banker & Rhodes, Informa Healthcare, 4^{th} ed. (2002); and “Goodman & Gilman's The Pharmaceutical Basis of Therapeutics,” McGraw-Hill, New York, 10th ed. (2001) can be consulted.

The DHA ester of the present invention is orally active and this route of administration can be used in the invention. Accordingly, administration forms can include, but are not limited to, tablets, dragees, capsules, caplets, gel caps, and pills, which contain the DHA ester and one or more suitable pharmaceutically acceptable carriers.

For oral administration, the DHA ester can be formulated readily by combining these compounds with pharmaceutically acceptable carriers well known in the art. Such carriers enable the compounds of the invention to be formulated as tablets, gel caps, pills, dragees, capsules, liquids, gels, syrups, slurries, suspensions and the like, for oral ingestion by a subject to be treated. In some embodiments, the dosage form is a tablet, gel cap, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, vegetable oil (e.g., soybean oil), and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations which can be used orally include, but are not limited to, push-fit capsules made of gelatin, as well as soft, sealed capsules made of gelatin and a plasticizer, such as glycerol or sorbitol. A preferred oral formulation is a soft gel cap. Capsule shells can be composed of non-animal derived ingredients, i.e., vegetarian ingredients, such as carrageenan, alginate, modified forms of starch, cellulose and/or other polysaccharides. All formulations for oral administration should be in dosages suitable for such administration.

By way of example, administration can be by parenteral, subcutaneous, intravenous (bolus or infusion), intramuscular, or intraperitoneal routes. Dosage forms for these modes of administration can include conventional forms, either as liquid solutions or suspensions, solid forms suitable for solution or suspension in liquid prior to injection, or as emulsions.

The present invention is also directed to oral dosage forms comprising: (a) about 200 mg to about 4 g of DHA ester, wherein the DHA ester is about 60% to about 99.5% (w/w) of the total fatty acid content of the dosage form; and (b) a pharmaceutically acceptable excipient, wherein the dosage form is substantially free of EPA, and wherein the DHA ester is derived from an algal source.

The present invention is also directed to a gel cap oral dosage form comprising an encapsulating material and an active, preferably a DHA ester of the invention. Optionally the gel cap also comprises a colorant, flavoring, and/or antioxidant.

The present invention includes gel caps that are hard or a soft gelatin capsules. In one embodiment, the encapsulating material comprises a gelatin, a plasticizer, and water. In certain embodiments, the encapsulating material is vegetarian, i.e., made from non-animal derived material, including plants, seaweed (for example, carrageenan), food starch, modified corn starch, potato starch, and tapioca. In other embodiments, the encapsulating material is derived from animals, including porcine, bovine and fish-based materials, such as gelatins. Plasticizers of the invention include glycerin, glycerol, polyols, and mixtures thereof. In some embodiments, the plasticizer is a high boiling point polyol, such as glycerol or sorbitol.

In one embodiment, the gel cap is a soft-gelatin capsule made from gelatin, glycerol and water, and filled with DHA-EE and an antioxidant. In certain embodiments, the gel cap is animal or vegetable derived. In one embodiment the gel cap is filled with DHA ester, preferably DHA-EE. In one embodiment, the gel cap comprises a 1 gram dosage form, wherein the fill weight of the dosage form is from about 950 to about 1050 mg, and wherein the gel cap contains from about 855 mg/g to about 945 mg/g DHA-ethyl ester.

In certain embodiments, the DHA esters of the invention comprise about 90%, about 91%, about 92%, from about 85.5% to about 94.5%, from about 90 to about 92%, or from about 87.5% to about 92.5% wt of the total fatty acid content of the dosage form. In one embodiment, the DHA esters of the invention comprise greater than, or greater than or equal to, about 90%, about 91%, or about 92% wt of the total fatty acid content of the dosage. Preferably the DHA esters of the invention comprise about 90% wt of the total fatty acid content of the dosage. In a one gram dosage form embodiment, the gel cap contains about 900 mg DHA per 1,000 g of the dosage. In one embodiment, the gel cap contains between about 900 mg to about 1 gram of DHA ester. In a preferred embodiment, the gel caps of the invention are administered in an amount up to a daily amount of 2 grams of DHA ester. In one embodiment, the DHA ester of the invention is derived from marine microalgae, preferably C. cohnii. In certain aspects of the invention, the gel caps are administered in daily dosage amounts, and regimens as described herein. In certain aspects of the invention, the gel caps are formulated in dosage amounts as described herein.

In some embodiments, the DHA esters of the invention are derived from undiluted oil from a single cell microorganism, and in one embodiment, from undiluted DHASCO-T® (Martek Biosciences Corporation). In some embodiments, the oil from which DHA esters of the invention are derived include single cell microorganism oils that are manufactured by a controlled fermentation process followed by oil extraction and purification using methods common to the vegetable oil industry. In certain embodiments, the oil extraction and purification steps include refining, bleaching and deodorizing. In one embodiment, the undiluted DHA oil contains about 40% to about 50% DHA by weight (about 400-500 mg DHA/g oil). In certain embodiments, the undiluted DHA oil is enriched by cold fractionation (resulting in oil containing about 60% w/w of DHA triglyceride), which DHA fraction optionally may then be transesterified, and subjected to further downstream processing to produce the active DHA of the invention, i.e., DHA esters. In some aspects of the invention, downstream processing of the oil comprises distillation and/or silica refinement.

Thus, to produce oil from which DHA esters of the invention are derived, in certain aspects of the invention, the following steps are used: fermentation of a DHA producing microorganism; harvesting the biomass; spray drying the biomass; extracting oil from the biomass; refining the oil; bleaching the oil; chill filtering the oil; deodorizing the oil; and adding an antioxidant to the oil. In one embodiment, the microorganism (e.g., Crypthecodinium cohnii) culture is progressively transferred from smaller scale fermenters to a production size fermenter. In come embodiments, following a controlled growth over a pre-established period, the culture is harvested by centrifugation then pasteurized and spray dried. In certain embodiments, the dried biomass is flushed with nitrogen and packaged before being stored frozen at −20° C. In certain embodiments, the DHA oil is extracted from the dried biomass by mixing the biomass with n-hexane or isohexane in a batch process which disrupts the cells and allows the oil and cellular debris to be separated. In certain embodiments, the solvent is then removed.

In one embodiment, the crude DHA oil then undergoes a refining process to remove free fatty acids and phospholipids. The refined DHA oil is transferred to a vacuum bleaching vessel to assist in removing any remaining polar compounds and pro-oxidant metals, and to break down lipid oxidation products. The refined and bleached DHA oil undergoes a final clarification step by chilling and filtering the oil to facilitate the removal of any remaining insoluble fats, waxes and solids.

Optionally, the DHA oil is deodorized under vacuum in a packed column, counter current steam stripping deodorizer. Antioxidants consisting of ascorbyl palmitate and natural mixed tocopherols may optionally be added to the deodorized oil to help stabilize the oil. In one embodiment, the final, undiluted DHA oil is maintained frozen at −20° C. until further processing. In one embodiment, the DHA oil has characteristics that fall within the limits set forth below in Table 1.

TABLE 1
Characteristics of Undiluted DHA Oil

| Test | Specification |
|---|---|
| DHA content, mg DHA/g oil | Min. 480 mg/g |
| Free Fatty Acid (FFA) | Max. 0.4% |
| Peroxide Value (PV) | Max. 5 meq/kg |
| Anisidine Value (AV) | Max 20 |
| Moisture and Volatiles (M & V) | Max. 0.02% |
| Unsaponifiable Matter | Max. 3.5% |
| Insoluble Impurities | Max. 0.1% |
| Trans Fatty Acid | Max. 1% |
| Arsenic | Max. 0.5 ppm |
| Cadmium | Max. 0.2 ppm |
| Chromium | Max. 0.2 ppm |
| Copper | Max. 0.1 ppm |
| Iron | Max. 0.5 ppm |
| Lead | Max. 0.2 ppm |
| Manganese | Max. 0.04 ppm |
| Mercury | Max. 0.04 ppm |
| Molybdenum | Max. 0.2 ppm |
| Nickel | Max. 0.2 ppm |
| Phosphorus | Max. 10 ppm |
| Silicon | Max. 500 ppm |
| Sulfur | Max. 100 ppm |
| 18:1 n-9 Oleic Acid | Max. 10% |
| 20:5 n-3 EPA | Max. 0.1% |
| Unknown Fatty Acids | Max. 3.0% |

In certain embodiments of the invention, the DHA oil is converted to DHA ester by methods known in the art. In one embodiment, DHA esters of the invention are produced from DHA oil by the following steps: cold fractionation and filtration of the DHA oil (to yield for example about 60% triglyceride oil); direct transesterification (to yield about 60% DHA ethyl ester); molecular distillation (to yield about 88% DHA ethyl ester); silica refinement (to yield about 90% DHA ethyl ester); and addition of an antioxidant.

In some embodiments, the cold fractionation step is carried out as follows: undiluted DHA oil (triglyceride) at ˜500 mg/g DHA is mixed with acetone and cooled at a controlled rate in a tank with −80° C. chilling capabilities. Saturated triglycerides crystallize out of solution, while polyunsaturated triglycerides at ˜600 mg/g DHA remain in the liquid state. The solids containing ˜300 mg/g DHA are filtered out with a 20 micron stainless steel screen from the liquid stream containing ˜600 mg/g DHA. The solids stream is then heated (melted) and collected. The 600 mg/g DHA liquid stream is desolventized with heat and vacuum and then transferred to the transesterification reactor.

In some embodiments, the transesterification step is carried out on the 600 mg/gm DHA oil, wherein that transesterification is done via direct transesterification using ethanol and sodium ethoxide. The transesterified material (DHA-EE) is then subject to molecular distillation and thus, further distilled (3 passes, heavies, lights, heavies) to remove most of the other saturated fatty acids and some sterols and non-saponifiable material. The DHA-EE is further refined by passing it though a silica column.

In one embodiment, the gel caps are 1 gram soft gelatin capsules, and have specifications within the limits set forth in Table 2:

TABLE 2
Sample Specifications for 1 gram DHA Ethyl Ester
Gel Caps of the Invention

| Test | Specification |
|---|---|
| DHA content, mg DHA/g oil | 855-945 mg/g |
| Ethyl Ester | Min. 90% esterified |
| Acid Value | Max. 2.0 mg KOH/g |
| Peroxide Value (PV) | Max. 10 meq/kg |
| Anisidine Value (AV) | Max. 20 |
| Arsenic | Max. 0.5 ppm |
| Copper | Max. 0.01 ppm |
| Iron | Max. 0.5 ppm |
| Lead | Max. 0.2 ppm |
| Mercury | Max. 0.04 ppm |
| Microbial Limits Test | Complies with <61> USP |

In one embodiment, the gel cap comprises a capsule preparation, an active, and optionally a colorant and/or an antioxidant. In another embodiment i) the capsule preparation comprises gelatin (bovine acid hide), glycerin, and purified water, ii) the active comprises DHA-EE, iii) the optional colorant is selected from titanium dioxide, FD&C Yellow #5, FD&C Red #40, and mixtures thereof; and iv) the antioxidant is ascorbyl palmitate. In one embodiment, the raw materials are USP raw materials.

In one embodiment, the gel caps are 1 gram soft gelatin capsules, and have the specifications within the limits set forth in Table 3:

TABLE 3
Sample Specifications for 1 gram DHA Ethyl Ester
Gel Caps of the Invention

| Test | Specification |
|---|---|
| DHA EE Content, per capsule | 855-945 mg |
| Average Fill Weight | 950-1050 mg |
| Disintegration | Complies USP |
| Acid Value | Max. 2.0 mg KOH/g |
| Peroxide Value (PV) | Max. 10 meq/kg |
| Anisidine Value (AV) | Max. 20 |
| Microbial Limits Tests | Complies with <61> USP |

Set forth in Table 4 is a list of components in one embodiment that are used in the manufacture of a DHA-EE soft gelatin capsule, and at least one corresponding function for each component.

TABLE 4
List of Components in 1 gram DHA Ethyl Ester
Soft Gelatin Capsules of the Invention

| Component | Function |
|---|---|
| 900 mg DHA EE | Active |
| Gelatin, Bovine Acid Hide | Capsule Preparation |
| Glycerin | Capsule Preparation |
| Purified Water | Capsule Preparation |
| Titanium Dioxide | Colorant |
| FD&C Yellow #5 | Colorant |
| FD&C Red #40 | Colorant |

In certain embodiments, the gel cap is vegetarian. In one embodiment, the capsule preparation contains no animal products, and comprises glycerol (and/or other polyols), seaweed extract (carrageenan) and water. In one embodiment, the water is purified. Optionally, in various embodiments color, flavor and/or sweeteners are added. During encapsulation, in one embodiment, fractionated coconut oil is used as a lubricant.

Administration of DHA esters according to the methods described herein can achieve a pharmacokinetic profile of DHA similar to that of a composition comprising DHA and EPA, e.g., Lovaza® (Reliant Pharmaceuticals), even though DHA ester of the present invention is substantially free of EPA. For example, absorption, incorporation into membranes, hydrolysis by esterases, absorption in the enterocytes, introduction into chylomicrons, very low density lipoproteins (VLDL), low density lipoproteins (LDL), and high density lipoproteins (HDL) of the DHA esters can be similar to that observed with a composition comprising DHA and EPA. In some embodiments, absorption, incorporation into membranes, hydrolysis by esterases, absorption in the enterocytes, introduction into chylomicrons, very low density lipoproteins (VLDL), low density lipoproteins (LDL), and high density lipoproteins (HDL) of the DHA esters can occur more rapidly relative to that observed with a composition comprising (a) DHA and EPA, (b) triglyceride form of DHA, and/or (c) an impure form of DHA (e.g., a composition wherein <79% of the total fatty acid composition is DHA). In some embodiments, the DHA ester is absorbed, incorporated into membranes, or hydrolyzed, absorbed into enterocytes, and/or introduction into chylomicrons, VLDL, LDL, and/or HDL at a rate 5%, 10%, 15% or 20% faster than that observed with a composition comprising (a) DHA and EPA, (b) triglyceride form of DHA, an/or (c) an impure form of DHA. In some embodiments, the DHA esters according to the methods described herein can achieve a reduction in triglyceride levels in a subject similar to that of a composition comprising DHA and EPA.

Retroconversion is an enzymatic process during which long-chain fatty acids are converted to their related shorter-chain precursor fatty acids though the incremental removal of two-carbon units from the molecule. DHA can be retroconverted to EPA and DPAn-3. See, e.g., Brossard et al., Am. J. Clin. Nutr. 64:577-86 (1996). In some embodiments, the DHA ester of the present invention is retroconverted to a lesser degree (or at a reduced rate) relative to DHA free acid and/or a salt form, or a DHA triglyceride form. For example, in some embodiments, less EPA and/or DPAn-3 is produced in the method using DHA esters of the present invention, relative to a method using a DHA free acid and/or salt form, or a DHA triglyceride form.

Metabolism of DHA esters can also result in the formation of Resolvin D1, Resolvin D2, Resolvin D3, and Resolvin D4. See, e.g., Serhan et al., Annu. Rev. Pathol. Mech Dis. 3:279-312 (2008). In some embodiments, the DHA ester metabolites have a similar pharmacokinetic profile to the DHA esters in, e.g., Lovaza®, even though DHA ester of the present invention is substantially free of EPA.

Administration of the DHA ester dosage forms of the present invention can be achieved using various regimens. For example, in some embodiments, administration of the DHA ester dosage forms is daily on consecutive days, or alternatively, the dosage form is administered every other day (bi-daily). Administration can occur on one or more days. For example, in some embodiments the DHA ester is administered daily for the duration of the subject's lifetime, or from 1 year to 20 years or 5 years to 10 years. In some embodiments, administration of the DHA ester dosage form occurs for 7, 14, 21, or 28 days. In some embodiments, administration of the DHA ester dosage form occurs until the triglyceride levels of the subject are lowered to a preselected target level, the target level being determined by a medical professional. In some embodiments, administration of the DHA ester dosage form continues even after the triglyceride levels of the subject have reached normal or borderline levels, or to a preselected target level. In some embodiments, the administration of the DHA ester is administered as a prophylactic measure, before the triglyceride levels become elevated.

Administration of DHA ester dosage forms can be combined with other regimens (i.e., non-DHA ester regimens) used to reduce triglyceride levels. For example, the method of the present invention can be combined with diet regimens (e.g., low carbohydrate diets, high protein diets, high fiber diets, etc.), exercise regimens, weight loss regimens, or smoking cessation regimens to lower triglyceride levels. The methods of the present invention can also be used in combination with other pharmaceutical products to lower triglyceride levels in a subject. Non-DHA ester regimens can also include other triglyceride-lowering pharmaceutical products including, e.g., bile acid binding resins, e.g., cholestyramine and cholestipol; niacin; fibric acid derivatives, e.g., gemfibozil and clofibrate; and statins, e.g., lovastatin, pravastatin, atorvastatin and simvastatin.

In some embodiments, the DHA esters of the present invention are administered before the non-DHA ester regimens. For example, the DHA ester dosage forms can be first used to reduce triglyceride levels, followed by administration of the non-DHA ester regimens to maintain (or further lower) the preselected triglyceride level. Alternatively, in some embodiments, the non-DHA ester regimens are administered first to lower the triglyceride levels in a subject to a preselected target level, and then the DHA ester dosage forms of the present invention are administered to maintain (or further lower) the lowered triglyceride levels in the subject. Thus, in some embodiments, the present invention is directed to a method of maintaining triglyceride levels using the DHA ester dosage forms of the present invention, the method comprising (1) administering a non-DHA ester regimen to a subject to lower the triglyceride levels in the subject, until the triglyceride levels have reached a preselected triglyceride level, and (2) administering the DHA ester dosage forms of the present invention to maintain the preselected triglyceride level. In some embodiments, the preselected triglyceride level is a triglyceride level in the normal triglyceride level range or the borderline high triglyceride level range.

The present invention is directed to kits or packages containing one or more dosage forms to be administered according to the methods of the present invention. A kit or package can contain one dosage form, or more than one dosage forms (i.e., multiple dosage forms). If multiple dosage forms are present in the kit or package, the multiple dosage forms can be optionally arranged for sequential administration. In some embodiments, the dosage forms are packaged in blister cards or blister packs. The kits can contain dosage forms of a sufficient number to provide convenient administration to a subject who has a chronic condition and requires long-term administration of the DHA ester of the present invention. Each dosage form can contain about 500 mg to about 4 g DHA ester and can be intended for ingestion on successive days. For example, in some embodiments, the kit provides dosage forms of a sufficient number for 1, 2, 3 or 4 months of daily administration of the DHA ester. In some embodiments of the present invention, the kit comprises dosage forms for shorter periods of administration, e.g., the kit can contain about 7, 14, 21, 28 or more dosage forms for oral administration, each dosage form containing about 500 mg to about 4 g DHA ester and intended for ingestion on successive days. The method of the present invention can include administration of the dosage form daily for extended periods of time, e.g., 6 months, 1 year, 18 months, 2 years, 5 years, 10 years, 20 years, or indefinitely for the duration of a subject's life. The method also can include administration of the dosage form daily for shorter periods of time, e.g., once daily for at least 7, 14, 21, or 28 consecutive days. In some embodiments, the invention is directed to a method of reducing plasma triglyceride level in a subject, the method comprising administering daily to the subject a dosage form comprising about 200 mg to about 4 g of DHA ester substantially free of EPA, wherein the dosage form is administered daily for 4 to 28 consecutive days, or for 7 to 14 consecutive days.

The kits of the present invention can optionally contain instructions associated with the dosage forms of the kits. Such instructions can be in a form prescribed by a governmental agency regulating the manufacture, use or sale of pharmaceutical products, which notice reflects approval by the agency of the manufacture, use or sale for human administration to treat a condition or disorder. The instructions can be in any form which conveys information on the use of the dosage forms in the kit according to the methods of the invention. For example, the instructions can be in the form of printed matter, or in the form of a pre-recorded media device.

In certain embodiments, during the course of examination of a patient, a medical professional will determine that administration of one of the methods of the present invention is appropriate for the patient, or the physician will determine that the patient's condition (e.g., the patient is suffering triglyceridemia) can be improved by the administration of one of the methods of the present invention. Prior to prescribing any DHA ester regimen, the physician can counsel the patient, for example, on the various risks and benefits associated with the regimen. The patient can be provided full disclosure of all the known and suspected risks associated with the regimen. Such counseling can be provided verbally, as well as in written form. In some embodiments, the physician can provide the patient with literature materials on the regimen, such as product information, educational materials, and the like.

The present invention is also directed to methods of educating consumers about the methods of lowering triglyceride levels of the present invention, the method comprising distributing the DHA ester dosage forms with consumer information at a point of sale. In some embodiments, the distribution will occur at a point of sale having a pharmacist or healthcare provider.

The term “consumer information” can include, but is not limited to, an English language text, non-English language text, visual image, chart, telephone recording, website, and access to a live customer service representative. In some embodiments, consumer information will provide directions for use of the DHA ester dosage forms according to the methods of the present invention, appropriate age use, indication, contraindications, appropriate dosing, warnings, telephone number of website address. In some embodiments, the method further comprises providing professional information to relevant persons in a position to answer consumer questions regarding use of the disclosed regimens according to the methods of the present invention. The term “professional information” includes, but is not limited to, information concerning the regimen when administered according to the methods of the present invention that is designed to enable a medical professional to answer customer questions.

A “medical professional,” includes, for example, a physician, physician assistant, nurse practitioner, pharmacist and customer service representative. All of the various aspects, embodiments and options described herein can be combined in any and all variations.

The following examples are illustrative, but not limiting, of the compositions and methods of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### EXAMPLES

### Example 1

### Purification of DHA Ethyl Ester from Algal Source

This example illustrates a method for purifying ethyl docosahexaenoate from docosahexaenoic acid-containing single cell oil.

150 mL of absolute ethanol (EtOH) was added to 175 g (approximately 0.2 moles of triglyceride) of DHASCO®-T oil (Martek Biosciences Corporation, Columbia, Md., having a DHA content of 0.4 g/g oil) in a one-liter flask under nitrogen (N₂) at room temperature. DHASC®-T oil is prepared from the microalgae Crypthecodinium cohnii. The mixture was allowed to stir for 15 minutes to obtain a homogeneous solution. Then 67 g of a 21% solution of sodium ethoxide/ethanol (NaOEt/EtOH; approximately 1.04 molar equivalents of triglycerides) was added to the solution and the mixture was allowed to reflux under N₂ for about 9 hours. The progress of the reaction was monitored by gas chromatography (GC) and thin-layer chromatography (TLC). When the reaction was completed, approximately 75 mL of EtOH was removed by distillation. The reaction mixture was then allowed to cool to room temperature under N₂. Hexane (300 mL) was added to the cooled reaction mixture, and the mixture was allowed to stir for 15 minutes at room temperature. Then 300 mL of deionized water was added to the mixture, and the mixture was allowed to stir for an additional 15 minutes. After removing and saving the organic layer, the aqueous layer was washed twice with 300 mL portions of hexane. A dark brown aqueous layer was discarded. The combined organic layers were then washed with 200 mL of a saturated NaCl solution. A GC analysis of the organic layer indicated the presence of about 44.7% DHA ethyl ester; the remaining materials were predominantly lower molecular weight ethyl esters (see Table 5).

The combined organic layer was concentrated under reduced pressure. The crude concentrate was then subjected to vacuum fractional distillation. The lower molecular weight ethyl esters were collected at temperatures between 100-150° C. and at a pressure of 0.8 mm Hg. The major components of this fraction were oleic, saturated C-14, and C-12 esters. The DHA ethyl ester was collected at temperatures between 155-165° C. and at a pressure of 0.8 mm Hg. A GC analysis of the DHA ethyl ester fraction showed a purity of about 91.3% DHA ethyl ester (see Table 5). From the fractional distillation, 68 g (86% yield) of the DHA ethyl ester was obtained as a light yellow oil.

TABLE 5
GC Analysis of DHASCO ®-T Oil Transesterification
and Distillation Products

| | | DHA Ethyl Ester-Containing |
|---|---|---|
| | Organic Layer After | Fraction After Vacuum |
| Sample | Transesterification | Fractional Distillation |
| % 22:6 (n-3) DHA | 44.72 | 91.29 |
| % 20:5 (n-3) EPA | 0.00 | 0.00 |
| % Additional | 55.28 | 8.81 |
| components | | |

### Example 2

### Purification of DHA Ethyl Ester

This example illustrates a method for purifying ethyl docosahexaneoate from a crude Crypthecodinium cohnii oil.

A crude oil obtained from Crypthecodinium cohnii by hexane extraction (DHA content of 0.5 g/g oil) was used directly without any further processing, such as winterization and/or RBD processing. Absolute ethanol (150 mL) was added to 175 g (approximately 0.2 moles of triglycerides) of the crude oil in a one-liter flask under N₂ at room temperature. The mixture was allowed to stir for 15 minutes to obtain a homogeneous solution. Then 67 g of a 21% solution of NaOEt/EtOH (approximately 1.04 molar equivalents of triglycerides) was added to the solution, and the mixture was allowed to reflux under N₂ for about 10 hours. The progress of the reaction was monitored by GC and TLC. When the reaction was completed, approximately 75 mL of ethanol was removed by distillation, and the mixture was allowed to cool to room temperature under N₂. Hexane (300 mL) was added to the cooled mixture, and the mixture was allowed to stir for 15 minutes at room temperature. 300 mL of deionized water was then added to the mixture, and the mixture was allowed to stir for an additional 15 minutes. After removing and saving the organic layer, the aqueous layer was washed twice with 300 mL portions of hexane. The combined organic layer was then washed with 200 mL of a saturated NaCl solution. A GC analysis of the organic layer indicated the presence of about 51% DHA ethyl ester; the remaining materials were predominantly lower molecular weight ethyl esters (see Table 6).

The combined organic layer was concentrated under reduced pressure. The crude concentrate was then subjected to vacuum fractional distillation. The lower molecular weight ethyl esters were collected at temperatures between 100-150° C. and at a pressure of 0.8 mm Hg. The major components of this fraction were oleic, saturated C-14, and C-12 esters. The DHA ethyl ester was collected at temperatures between 155-165° C. and at a pressure of 0.8 mm Hg. A GC analysis of the DHA ethyl ester fraction showed a purity of about 92% DHA (see Table 6). From the fractional distillation, 69 g (66% yield) of the DHA ethyl ester was obtained as a light yellow oil.

TABLE 6
GC Analysis of Crude Crypthecodinium cohnii Oil
Transesterification and Distillation Products

| | | DHA Ethyl Ester-Containing |
|---|---|---|
| | Organic Layer After | Fraction After Vacuum |
| Sample | Transesterification | Fractional Distillation |
| % 22:6 (n-3) DHA | 51.25 | 91.80 |
| % 20:5 (n-3) EPA | 0.00 | 0.00 |
| % Additional | 48.75 | 8.20 |
| components | | |

### Example 3

### Effect of DHA Ethyl Ester on Triglyceride Levels

The effect of the DHA ethyl ester (DHA-EE) produced according to the method of Example 2 on triglyceride levels was investigated using male Wister rats. The DHA-EE used in this example contained DHA at about 93.6% (w/w) of the total fatty acid content of the dosage form. The effect of purified DHA-EE was compared to two different DHA-containing products: DHASCO-T® (Martek Bioscience Corporation, Columbia, Md.) and Lovaza® (Reliant Pharmaceuticals, Inc., Durham, N.C.). DHASCO-T® comprises approximately 45% DHA and 55% other fatty acids (with substantially no EPA). Lovaza® comprises approximately 41.7% DHA ethyl ester, 51.7% EPA ethyl ester, and 6.4% other fatty acids. The vehicle control comprised corn oil. 84 Wistar rats were randomized into 7 groups of 12 rats each. Each rat was administered orally a high fructose diet for 4-5 weeks to raise triglyceride levels. After 4-5 weeks, rats with a triglyceride level <300 mg/dL were excluded. Then each rat was administered either (a) a vehicle control, (b) DHA ethyl ester (0.6 g/kg/day, 1.3 g/kg/day, 2.5 g/kg/day, or 5.0 g/kg/day), (c) DHASCO-T (5.0 g/kg/day), or (d) Lovaza (5.0 g/kg/day) for 28 days by oral gavage. Diets were controlled for calories, nutrients and levels of vitamin E. Triglyceride levels of the rats were measured at 14 days and 28 days. The results are presented in Table 7. Triglyceride values shown are averages for each treatment group, presented as actual mean values.

TABLE 7
Effect of DHA-EE, DHASCO-T, and Lovaza on Mean Triglyceride Levels^{a}

| | | Control | DHA-EE | | | | DHASCO-T ® | LOVAZA ® |
|---|---|---|---|---|---|---|---|---|
| | g/kg/day | 0.0 | 0.6 | 1.3 | 2.5 | 5.0 | 5.0 | 5.0 |
| | fatty acid | | | | | | | |
| | g/kg/day | 0.0 | 0.56 | 1.2 | 2.3 | 4.7 | 2.25 | 2.09 |
| | DHA (approx) | | | | | | | |
| 14 | Triglycerides | 263.6 ± 100.3 | 176.5 ± 96.4^{†} | 175.2 ± 50.6^{†} | 115.5 ± 60.9^{†} | 97.6 ± 47.8^{†} | 120.8 ± 36.8^{†} | 125.2 ± 73.8^{†} |
| day | mg/dL | | | | | | | |
| | % of control | 100 | 67 | 66 | 44 | 37 | 46 | 47 |
| 28 | Triglycerides | 192.2 ± 65.4 | 182.7 ± 86.9^{‡} | 114.7 ± 37.2^{†} | 107.9 ± 58.4^{†} | 78.9 ± 46.5^{†} | 122.6 ± 57.2 | 70.0 ± 28.9^{†} |
| day | mg/dL | | | | | | | |
| | % of control | 100 | 95 | 60 | 56 | 41 | 64 | 36 |

^{a}Data are presented as mean ± SD.
^{†}P < 0.05 vs control.
^{‡}P < 0.05 vs Lovaza.

The triglyceride results presented below in Table 8 are calculated from the same data set as in Table 7. Triglyceride values shown in Table 8 are averages for each treatment group, presented as least squares mean values.

TABLE 8
Effect of DHA-EE, DHASCO, and Lovaza on LSMEAN Serum Triglyceride Levels^{a}

| | | Control | DHA-EE | | | | DHASCO-T ® | LOVAZA ® |
|---|---|---|---|---|---|---|---|---|
| | g/kg/day | 0.0 | 0.6 | 1.3 | 2.5 | 5.0 | 5.0 | 5.0 |
| | fatty acid | | | | | | | |
| | g/kg/day | 0.0 | 0.56 | 1.2 | 2.3 | 4.7 | 2.25 | 2.09 |
| | DHA (approx) | | | | | | | |
| 14 | Triglycerides | 258.5 | 168.4† | 173.6† | 120.2† | 110.7† | 123.8† | 120.6† |
| day | mg/dL | | | | | | | |
| | % of control | 100 | 65 | 67 | 47 | 43 | 48 | 47 |
| 28 | Triglycerides | 187.1 | 174.5 | 113.1† | 112.6† | 87.6† | 125.6 | 62.9† |
| day | mg/dL | | | | | | | |
| | % of control | 100 | 93 | 60 | 60 | 47 | 67 | 35 |

^{a}Data are presented as LSMEAN.
†P < 0.05 vs control.
‡P < 0.05 vs Lovaza.

The data suggests that DHA ethyl ester (DHA-EE) reduces triglyceride levels at 14 days, even at the lowest dosage level (0.6 g/kg/day). The DHA ethyl ester reduces triglyceride levels, even in the absence of EPA. At day 28, 0.6 g DHA-EE and DHASCO-T® were not significantly lower than control. Thus, reduced amounts of DHA ethyl ester (without EPA) can be used to achieve lowered triglyceride levels similar to the dosage amounts commonly assigned to Lovaza® and DHASCO-T®.

### Example 4

### Effect of DHA Ethyl Ester on Cholesterol Levels

The effect of the DHA ethyl ester of Example 2 on cholesterol levels was investigated using male Wister rats, and was compared to DHASCO-T® and Lovaza®. The rats were administered either (a) a vehicle control, (b) DHA ethyl ester (0.6 g/kg/day, 1.3 g/kg/day, 2.5 g/kg/day, or 5.0 g/kg/day), (c) DHASCO-T (5.0 g/kg/day), or (d) Lovaza (5.0 g/kg/day) for 28 days. Cholesterol levels, i.e., total cholesterol, of the rats were measured at 14 days and 28 days. The results are presented in Table 8. Total cholesterol values shown are averages for each treatment group, i.e., actual mean values.

TABLE 9
Effect of DHA-EE, DHASCO, and Lovaza on Mean Serum Cholesterol Levels^{a}

| | | Control | DHA-EE | | | | DHASCO-T ® | LOVAZA ® |
|---|---|---|---|---|---|---|---|---|
| | g/kg/day | 0.0 | 0.6 | 1.3 | 2.5 | 5.0 | 5.0 | 5.0 |
| | fatty acid | | | | | | | |
| | g/kg/day | 0.0 | 0.56 | 1.2 | 2.3 | 4.7 | 2.25 | 2.09 |
| | DHA (approx) | | | | | | | |
| 14 day | Cholesterol | 91.1 ± 15.5 | 72.2 ± 17.7 | 75.0 ± 24.0 | 77.6 ± 23.1 | 67.1 ± 17.9 | 63.0 ± 20.5 | 61.4 ± 11.3 |
| | mg/dL | | | | | | | |
| | % of control | 100 | 79 | 82 | 85 | 74 | 69 | 67 |
| 28 day | Cholesterol | 100.3 ± 47.0 | 80.3 ± 21.8 | 62.0 ± 16.0† | 71.5 ± 17.8† | 60.2 ± 19.3† | 87.2 ± 30.8 | 65.6 ± 18.4 |
| | mg/dL | | | | | | | |
| | % of control | 100 | 80 | 62 | 72 | 60 | 87 | 66 |

^{a}Data are presented as mean ± SD
†P < 0.05 vs control
‡P < 0.05 vs Lovaza

The cholesterol results presented below in Table 10 are calculated from the same data set as in Table 9. Cholesterol level values shown in Table 10 are averages for each treatment group, presented as least squares mean values.

TABLE 10
Effect of DHA-EE, DHASCO, and Lovaza on LSMEAN Serum Cholesterol Levels^{a}

| | | Control | DHA-EE | | | | DHASCO-T ® | LOVAZA ® |
|---|---|---|---|---|---|---|---|---|
| | g/kg/day | 0.0 | 0.6 | 1.3 | 2.5 | 5.0 | 5.0 | 5.0 |
| | fatty acid | | | | | | | |
| | g/kg/day | 0.0 | 0.56 | 1.2 | 2.3 | 4.7 | 2.25 | 2.09 |
| | DHA (approx) | | | | | | | |
| 14 day | Cholesterol | 90.7 | 72.4 | 74.6 | 77.4 | 67.8 | 63.2 | 61.8 |
| | mg/dL | | | | | | | |
| | % of control | 100 | 80 | 82 | 85 | 75 | 69 | 68 |
| 28 day | Cholesterol | 100.0 | 80.6 | 61.6† | 71.3† | 60.9† | 87.4 | 65.9 |
| | mg/dL | | | | | | | |
| | % of control | 100 | 80 | 62 | 72 | 60 | 87 | 66 |

^{a}Data are presented as LSMEAN
†P < 0.05 vs control
‡P < 0.05 vs Lovaza

The data suggests that DHA-EE reduces cholesterol levels by 14 days, even at the lowest dosage level. The DHA-EE reduces cholesterol levels, even in the absence of EPA. Thus, reduced amounts of DHA (without EPA) can be used to achieve cholesterol levels significantly lower than the dosage amounts commonly assigned to Lovaza® and DHASCO-T®.

### Example 5

### Plasma Levels of DHA Ethyl Ester

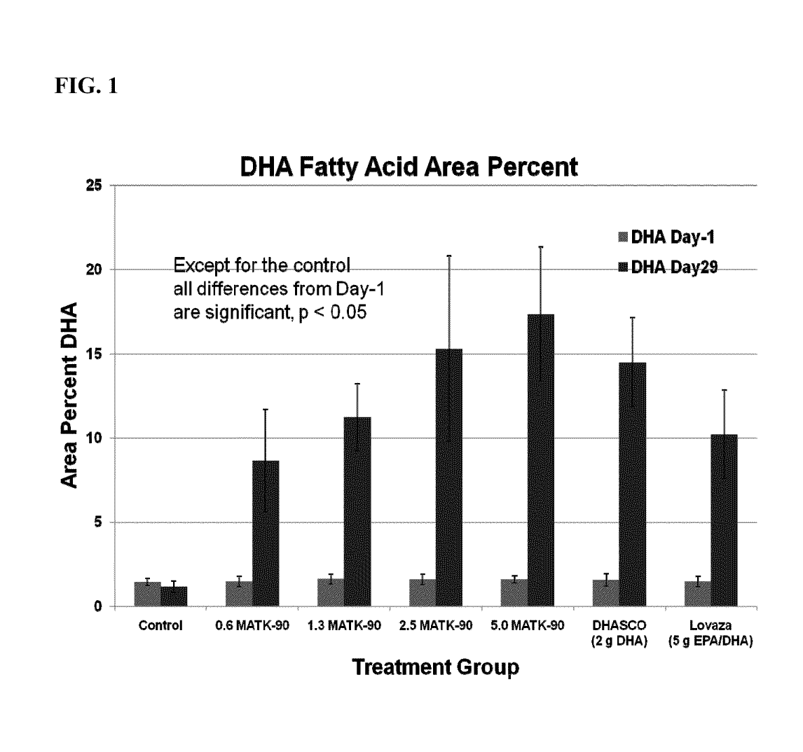

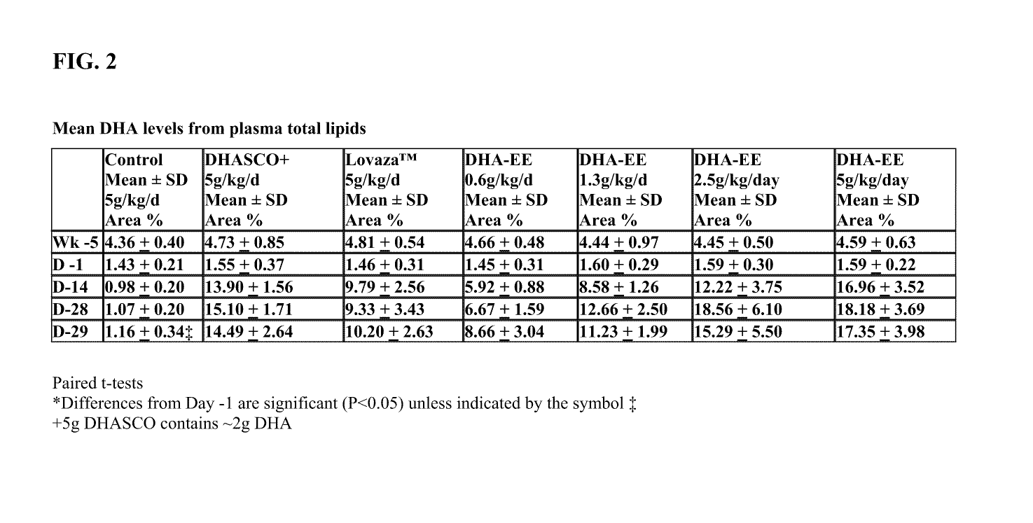

The plasma DHA fatty acid area percent and plasma EPA fatty acid area percent were determine in the rats at day 1 and day 29 (post necropsy). FIG. 1 demonstrates that plasma DHA fatty acid area percent correlates with increasing dosing of DHA, either from purified DHA-EE, DHASCO®, or Lovaza®. FIG. 2 demonstrates that plasma EPA fatty acid area percent does not increase in rats administered DHA-EE or DHASCO® to the same extent that it is increased in Lovaza®.

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications and patent applications mentioned in this specification are herein incorporated by reference to the same extent as if each individual publication or patent application was specifically and individually indicated to be incorporated by reference in its entirety.

What is claimed is:

1. A method of reducing plasma triglyceride level in a human subject, the method comprising administering daily an oral dosage form comprising docosahexaenoic acid (DHA) ester to a subject in need thereof, wherein less than 3% (w/w) of the total fatty acid content of the dosage form is eicosapentaenoic acid (EPA), wherein the amount of DHA ester in the dosage form is between about 450 mg and about 2.0 grams, and wherein the DHA ester is derived from an algal source.

2. A method of reducing plasma triglyceride level in a human subject, the method comprising administering daily to the subject an oral dosage form comprising docosahexaenoic acid (DHA) to a subject in need thereof, wherein less than 3% (w/w) of the total fatty acid content of the dosage form is eicosapentaenoic acid (EPA), wherein the DHA ester is from about 60% to about 99.5% (w/w) of the total fatty acid content of the dosage form, and wherein the amount of DHA ester in the dosage form is between about 450 mg and about 2.0 grams.

3. A method of reducing plasma triglyceride level in a human subject, the method comprising administering daily to the subject an oral dosage form comprising from about 200 mg to about 3 grams of docosahexaenoic acid (DHA) ester to a subject in need thereof, wherein less than 3% (w/w) of the total fatty acid content of the dosage form is eicosapentaenoic acid (EPA).

4. The method of any one of claims 1 to 3, wherein the DHA ester is a DHA alkyl ester.

5. The method of claim 4, wherein the DHA alkyl ester is a DHA methyl ester, ethyl ester, or propyl ester.

6. The method of claim 2 or 3, wherein the DHA ester is derived from an algal source.

7. The method of any one of claims 1 to 3, wherein the algal source is Crypthecodinium cohnii or Schizochytrium sp.

8. The method of any one of claims 1 and 3, wherein the DHA ester is from about 60% to about 99.5% (w/w) of the total fatty acid content of the dosage form.

9. The method of claim 8, wherein the DHA ester is greater than or equal to about 90% (w/w) of the total fatty acid content of the dosage form.

10. The method of any one of claims 1 to 3, wherein the dosage form comprises about 450 mg, about 500 mg, about 900 mg, or about 1 g of DHA ester.

11. The method of any one of claims 1 to 3, wherein the dosage form comprises from 450 about mg or about 900 mg of DHA ester.

12. The method of any one of claims 1 to 3, wherein the dosage form comprises 450 mg DHA ester.

13. The method of any one of claims 1 to 3, wherein the dosage form comprises 900 mg DHA ester.

14. The method of any one of claims 1 to 3, wherein the EPA is less than 1% of the total fatty acid content of the dosage form.

15. The method of any one of claims 1 to 3, wherein the EPA is less than 0.2% of the total fatty acid content of the dosage form.

16. The method of any one of claims 1 to 3, wherein the EPA is less than 0.01% of the total fatty acid content of the dosage form.

17. The method of any one of claims 1 to 3, wherein the EPA is not detected in the dosage form.

18. The method of any one of claims 1 to 3, wherein the dosage form comprises from about 0.1% to 20% of one or more of the following fatty acids, or esters thereof:
(a) capric acid;
(b) lauric acid;
(c) myristic acid;
(d) palmitic acid
(e) palmitoleic acid;
(f) stearic acid;
(g) oleic acid;
(h) linoleic acid;
(i) a-linolenic acid;
(j) docosapentaenoic acid 22:5n-3, 22:5w3 (DPAn3);
(k) docosapentaenoic acid 22:5n-6, 22:5w6 (DPAn6); and
(l) 4,7,10,13,16,19,22,25 octacosaoctaenoic acid (C28:8).

19. The method of any one of claims 1 to 3, wherein the dosage form comprises from 1% to 5% of one or more of the following fatty acids, or esters thereof:
(a) capric acid;
(b) lauric acid;
(c) myristic acid;
(d) palmitic acid
(e) palmitoleic acid;
(f) stearic acid;
(g) oleic acid;
(h) linoleic acid;
(i) α-linolenic acid;
(j) docosapentaenoic acid 22:5n-3, 22:5w3 (DPAn3);
(k) docosapentaenoic acid 22:5n-6, 22:5w6 (DPAn6); and
(l) 4,7,10,13,16,19,22,25 octacosaoctaenoic acid (C28:8).

20. The method of any one of claims 1 to 3, wherein the dosage form comprises less than 1% each of the following fatty acids, or esters thereof:
(a) capric acid;
(b) lauric acid;
(c) myristic acid;
(d) palmitic acid
(e) palmitoleic acid;
(f) stearic acid;
(g) oleic acid;
(h) linoleic acid;
(i) α-linolenic acid;
(j) docosapentaenoic acid 22:5n-3, 22:5w3 (DPAn3);
(k) docosapentaenoic acid 22:5n-6, 22:5w6 (DPAn6); and
(l) 4,7,10,13,16,19,22,25 octacosaoctaenoic acid (C28:8).

21. The method of any one of claims 1 to 3, wherein the dosage form is administered daily for the remainder of the subject's lifetime.

22. The method of any one of claims 1 to 3, wherein the dosage form is administered daily for 1 to 10 years.

23. The method of any one of claims 1 to 3, wherein the dosage form is administered daily for 4 to 28 consecutive days.

24. The method of claim 23, wherein the dosage form is administered daily for 7 to 14 consecutive days.

25. The method of claim 24, wherein the triglyceride level in the subject is reduced about 25% to about 75% by day 14.

26. The method of any one of claims 1 to 3, wherein the dosage form is administered at least once daily.

27. The method of any one of claims 1 to 3 wherein the dosage form is administered at least twice per day.

28. The method of any one of claims 1 to 3, wherein the dosage form comprising DHA is administered in a combination regimen with a statin.

29. The method of any one of claims 1 to 3, wherein the oral dosage form is a tablet, pill, gel cap, or caplet.

30. An oral dosage form comprising:
(a) from about 200 mg to about 1 gram of DHA ester; wherein the DHA ester is greater than or equal to about 90% (w/w) of the total fatty acid content of the dosage form; and
(b) a pharmaceutically acceptable excipient;
wherein less than 3% (w/w) of the total fatty acid content of the dosage form is eicosapentaenoic acid (EPA).

31. The dosage form of claim 30, wherein the dosage form is a gel cap comprising an active and a capsule preparation, wherein the active comprises DHA ethyl ester, wherein less than 3% (w/w) of the total fatty acid content of the dosage form is eicosapentaenoic acid.

32. The dosage form of claim 31 wherein the capsule preparation comprises a plasticizer, gelatin, and water.

33. The dosage form of claim 32 wherein the plasticizer is glycerine or glycerol.

34. The dosage form of claim 30 wherein the amount of DHA ester in the dosage form is 450 mg or 900 mg.

35. The dosage form of claim 30 wherein the amount of DHA ester in the dosage form is 450 mg.

36. The dosage form of claim 30 wherein the amount of DHA ester in the dosage form is 900 mg.
